SEQUENCE LISTING (11 sequences)

SEQ ID NO: 1 - DNA, 51 bp, Homo sapiens
  misc_feature (26)..(26): SNPrs9380548; n is a or t
cttctttggatttgcagtgcctacangactctcacagcgctactaggagct

SEQ ID NO: 2 - DNA, 51 bp, Homo sapiens
  misc_feature (26)..(26): SNPrs742490; n is g or c
tggccgatgctgtaagctccagtttnaatgccagccggctcgagacctaag

SEQ ID NO: 3 - DNA, 51 bp, Homo sapiens
  misc_feature (26)..(26): SNPrs7634; n is a or t
aaaagtagtttgaatgttttgtgtgnaaggagtataccatgagatgagatg

SEQ ID NO: 4 - DNA, 51 bp, Homo sapiens
  misc_feature (26)..(26): SNPrs1250301; n is g or a
gcctgggcgagagagtggatccttgnctctaaaaaataaatttataaaatc

SEQ ID NO: 5 - DNA, 51 bp, Homo sapiens
  misc_feature (26)..(26): SNPrs2797915;n is  t or c
cttgttcaggctgtgaggcaactaanattgtaaccatttgtaagtgtgcag

SEQ ID NO: 6 - DNA, 51 bp, Homo sapiens
  misc_feature (26)..(26): SNPrs4130857; n is a or g
gaaggaaaaagaaagaaaggacactnagtttcaaatctgtccctgagtcat

SEQ ID NO: 7 - DNA, 51 bp, Homo sapiens
  misc_feature (26)..(26): SNPrs4585205; n is t or c
tgcgatacctagaataatgttactgnccagaagaaaagtccaatacacaaa

SEQ ID NO: 8 - DNA, 51 bp, Homo sapiens
  misc_feature (26)..(26): SNPrs4602399; n is g or t
tgtgtaccaatttactatactagtcncttctcacactgctataaaaaactg

SEQ ID NO: 9 - DNA, 51 bp, Homo sapiens
  misc_feature (26)..(26): SNPrs4286440; n is  g or t
agaacattggaaaaatactaggatancttgcctctaatcttagctttatgt

SEQ ID NO: 10 - DNA, 51 bp, Homo sapiens
  misc_feature (26)..(26): SNPrs16867137; n is c or t
gaactctgtctagctccacccatttntaagttaggctgaaagacaggttaa

SEQ ID NO: 11 - DNA, 51 bp, Homo sapiens
  misc_feature (26)..(26): SNPrs1250302; n is g or a
catacagggaattcagtattggaaangggttgaatgaggtgacttcgaggg

(19) United States
(12) Patent Application Publication
(10) Pub. No.: US 2020/0095638 A1
(43) Pub. Date: Mar. 26, 2020

(54) METHODS FOR IDENTIFYING AND TREATING KAWASAKI DISEASE PATIENTS WITH INTRAVENOUS IMMUNOGLOBULIN RESISTANCE

(71) Applicant: KAOHSIUNG CHANG GUNG MEMORIAL HOSPITAL, Kaohsiung City (TW)

(72) Inventors: Ho-Chang KUO, Kaohsiung City (TW); Kai-Sheng HSIEH, Kaohsiung City (TW); Wei Chiao CHANG, Taipei (TW); Henry Sung-Ching WONG, Taipei (TW)

(21) Appl. No.: 16/141,371

(22) Filed: Sep. 25, 2018

(51) Int. Cl. C12Q 1/6883; C07K 16/00; A61K 39/395; A61K 45/06

(52) U.S. Cl. CPC C12Q 1/6883; C07K 16/00; C12Q 2600/106; A61K 45/06; A61K 39/395

(57) ABSTRACT

Methods for identifying Kawasaki Disease subjects resistant to intravenous immunoglobulin (IVIG) are provided, comprising the step of detecting at least one risk allele of the SNPs disclosed herein or calculating a weighted genetic risk score based on the presence of the risk alleles. Also provided are methods of treating Kawasaki Disease subjects with IVIG resistance, by administering a first dose of IVIG and an adjunctive therapy.

### BACKGROUND OF THE INVENTION

Kawasaki disease (KD) is an acute systemic vasculitis syndrome. This disease mainly affects children who are younger than 5 years of age. KD is thought to be the leading cause of acquired heart disease in children in the developed countries. High-dose intravenous immunoglobulin (IVIG) at 2 g/kg is currently acknowledged to be the standard treatment for KD patients. Receiving a single high dose of IVIG within 10 days after disease onset can significantly reduce the risk of coronary artery lesions (CALs) from 49.0% to 18.4%. However, 10% to 20% of KD patients have recrudescent fever after the initial IVIG treatment, possible causes include intercurrent infection or IVIG resistance. Patients resistant to IVIG have a higher risk of CAL and acquired cardiac complications in adulthood.

There is an unmet need for identify potentially IVIG resistant or refractory KD patients at the time of diagnosis and treating IVIG resistant KD patients with adjunctive therapy to reduce the risk of coronary artery lesions. The present invention addresses these and other needs.

### BRIEF SUMMARY OF THE INVENTION

In one embodiment, the present invention discloses method for treating Kawasaki Disease in a subject, comprising the steps of (a) detecting the presence of the risk allele of at least one single nucleotide polymorphism (SNP) selected from rs9380548, rs742490, rs7634, rs1250301, rs2797915, rs4130857, rs4585205, rs4602399, rs4286440, rs16867137 or rs1250302 in the sample of the subject; (b) identifying the subject with at least one risk allele in step (a) as resistant to IVIG; and (c) administering a first dose of IVIG and an adjunctive therapy to the subject.

In another embodiment, the present invention discloses methods treating Kawasaki Disease in a subject, comprising the steps of (a) detecting the presence of the risk alleles of the single nucleotide polymorphisms (SNPs) in the sample of the subject, said SNPs are selected from:

(i) rs9380548;

(ii) rs742490;

(iii) rs7634;

(iv) at least one SNP selected from rs1250301 or rs2797915;

(v) at least one SNP selected from rs4130857, rs4585205 or rs4602399;

(vi) rs4286440;

(vii) rs16867137; and

(viii) rs1250302;

(b) calculating a weighted genetic risk score (wGRS) based on the following formula;

$$wGRS = {\sum }_{k = 1}^{n} {w}^{k} {X}^{k} \qquad ( the wGRS formula )$$

wherein n is an integer between 8 to 11, k is the SNP of step (a), w^{k} is the corresponding weight of SNP (ln(OR)) and X^{k} is the number of the risk allele of the SNP (0, 1, or 2); and (c) identifying the subject as IVIG resistant if the wGRS in step (b) is equal to or higher than 0.01, and (d) administering a first dose of IVIG and an adjunctive therapy to the subject.

The present invention also discloses the use of a kit to identify a Kawasaki Disease subject resistant to IVIG the kit comprises an agent for detecting the risk allele of at least one SNP selected from rs9380548, rs742490, rs7634, rs1250301, rs2797915, rs4130857, rs4585205, rs4602399, rs4286440, rs16867137 or rs1250302 in the sample of the subject.

The present invention also provides methods to identify a Kawasaki Disease subject resistant to IVIG comprising the steps of (a) detecting the presence of the risk allele of at least one SNP selected from rs9380548, rs742490, rs7634, rs1250301, rs2797915, rs4130857, rs4585205 or rs4602399 in the sample of the subject, and (b) identifying the subject with at least one risk allele in step (a) as resistant to IVIG

Further provided are methods to identify a Kawasaki Disease subject resistant to IVIG comprising the steps of (a) detecting the presence of the risk allele of the single nucleotide polymorphisms (SNPs) in the sample of the subject, said SNPs are selected from:

(i) rs9380548;

(ii) rs742490;

(iii) rs7634;

(iv) at least one SNP selected from rs1250301 or rs2797915;

(v) at least one SNP selected from rs4130857, rs4585205 or rs4602399;

(vi) rs4286440;

(vii) rs16867137; and

(viii) rs1250302;

(b) calculating a weighted genetic risk score (wGRS) based on the wGRS formula described herein; and (c) identifying the subject as IVIG resistant if the wGRS in step (b) is equal to or higher than 0.01.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### DETAILED DESCRIPTION OF THE INVENTION

As used herein, the articles “a” and “an” refer to one or more than one (i.e., at least one) of the grammatical object of the article.

The term “subject” and “patient” may be used interchangeably and refer to a mammal diagnosed with KD or suspected of having KD. Subjects include primate, and more preferably, a human.

As used herein interchangeably, “IVIG-resistance,” “IVIG resistant” or “refractory to IVIG” are defined herein as fever (temperature above 38° C.) for 48 hours or more after the administration of first IVIG

All numbers herein may be understood as modified by “about.” As used herein, the term “about” is meant to encompass variations of ±10%.

Multiple single nucleotide polymorphisms (SNPs) for identifying KD patients refractory to or resistant to WIG according to an embodiment of the present invention include at least one polynucleotide sequences of SEQ ID NOS: 1 to 11,

TABLE 1

| Gene Bank accession | Polynucleotide | |
|---|---|---|
| of SNP in NCBI | Containing SNP | ln(OR) of the SNP |
| rs9380548 | SEQ ID NO: 1 | ln (1.630504) = 0.49 |
| rs7634 | SEQ ID NO: 2 | ln(1.267626) = 0.24 |
| rs742490 | SEQ ID NO: 3 | ln(1.489495) = 0.40 |
| rs1250301 | SEQ ID NO: 4 | ln(1.463538) = 0.38 |
| rs2797915 | SEQ ID NO: 5 | ln(1.463538) = 0.38 |
| rs4130857 | SEQ ID NO: 6 | ln(1.265988) = 0.24 |
| rs4585205 | SEQ ID NO: 7 | ln(1.265988) = 0.24 |
| rs4602399 | SEQ ID NO: 8 | ln(1.265988) = 0.24 |
| rs4286440 | SEQ ID NO: 9 | ln(1.261098) = 0.23 |
| rs16867137 | SEQ ID NO: 10 | ln(1.374688) = 0.32 |
| rs1250302 | SEQ ID NO: 11 | ln(1.43344) = 0.36 |

The risk alleles of the SNPs are described in Table 2. rs4130857, rs4585205, and rs4602399 on chromosome 3 and rs1250301 and rs2797915 on chromosome 10 are in highly linkage disequilibrium.

The GenBank accession No. of an SNP in the National Center for Biotechnology Information (NCBI) database indicates a sequence and a position of the SNP. Those skilled in the art may easily identify the sequence and the position of the SNP using the GenBank accession No. The specific sequences corresponding to the rs No. of the SNP registered in NCBI may change over time. It is obvious to those skilled in the art that the sequences are within the scope of the present invention, even if the corresponding rs number changes. The nucleotide sequences of SEQ ID NOS: 1 to 11 are polymorphic sequences. A polymorphic sequence is a polynucleotide sequence including a polymorphic site representing SNP. The polynucleotide sequences can be DNA or RNA.

Methods for Identifying KD Patients with IVIG Resistance

The identifying method includes isolating DNA from the sample of a patient with KD or suspect of having KD, determining a base sequence at a polymorphic site of the DNA, and judging that the patient is resistant to IVIG when the base sequence includes at least one SNP selected from SEQ ID NO:1 to SEQ ID NO:11 or at least one risk allele listed in Table 2.

In one embodiment, methods to identify a KD patient resistant to IVIG are provided, said methods comprise the steps of a) detecting the presence of the risk allele of at least one single nucleotide polymorphism (SNP) selected from rs9380548, rs742490, rs7634, rs1250301, rs2797915, rs4130857, rs4585205, rs4602399, rs4286440, rs16867137 or rs1250302 in the sample of the subject; and b) identifying the subject with at least one risk allele in step (a) as resistant to IVIG

In another embodiment, methods to identify a KD patient resistant to IVIG are provided, said methods comprise the steps of: (a) detecting the presence of the risk allele of the single nucleotide polymorphisms (SNPs) in the sample of the subject, said SNPs are selected from:

(i) rs9380548,

(ii) rs742490,

(iii) rs7634,

(iv) at least one SNP selected from rs1250301 or rs2797915,

(v) at least one SNP selected from rs4130857, rs4585205 or rs4602399,

(vi) rs4286440,

(vii) rs16867137, and

(viii) rs1250302;

(b) calculating a weighted genetic risk score (wGRS) based on the following formula:

$$wGRS = {\sum }_{k = 1}^{n} {w}^{k} {X}^{k} \qquad ( the wGRS formula )$$

wherein n is an integer between 8 to 11, k is the SNP of step (a), w^{k} is the corresponding weight of SNP (ln(OR)) and X^{k} is the number of the risk allele of the SNP (0, 1, or 2); and (c) identifying the subject as IVIG resistant if the wGRS in step (b) is equal to or higher than 0.01, 0.02, 0.03, 0.04, 0.05, 0.06, 0.07, 0.08, 0.09, 0.1, 0.2, 0.4, 0.5, 0.6, 0.7, 0.8, 0.9, 1.0, 1.1, 1.2. 1.3, 1.4, 1.5, 1.6 or 1.65. In an exemplary embodiment, the corresponding weights of SNPs (ln(OR)) are listed in Table 1.

The use of wGRS to aggregate the odds ratio of the SNPs of the present application significantly improved the predictive accuracy of IVIG resistance.

Some embodiments of the present invention are directed to methods of assessing whether a KD subject has, or is at risk of having IVIG resistance based on the identification of particular SNP in a test sample. Non limiting examples of the sample include DNA from peripheral white blood cells, serum, cell, tissue, or biopsy.

The polymorphisms can be detected by any available method, including amplification, hybridization to a probe, microarray analysis, real-time PCR, sequencing or the like, see B Sobrino et al, SNPs in forensic genetics: a review on SNP typing methodologies, Forensic Science International, Volume 154, Issues 2-3, 25 Nov. 2005, Pages 181-194. In one specific embodiment, SNP detection includes amplifying the polymorphism, linked locus or a sequence associated therewith (e.g., flanking sequences, transcribed sequences or the like) and detecting the resulting amplicon. For example, in one embodiment, amplifying includes a) admixing an amplification primer or amplification primer pair with a nucleic acid template isolated from the organism or biological sample. The primer or primer pair can be complementary or partially complementary to a region proximal to or including the polymorphism or linked locus, and are capable of initiating nucleic acid polymerization by a polymerase on the nucleic acid template. The primer or primer pair is extended in a DNA polymerization reaction comprising a polymerase and the template nucleic acid to generate the amplicon. In certain aspects, the amplicon is optionally detected by a process that includes hybridizing the amplicon to an array, digesting the amplicon with a restriction enzyme, or real-time PCR analysis. Optionally, the amplicon can be fully or partially sequenced, e.g., by hybridization. Typically, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other technologies can be substituted for amplification, e.g., use of branched DNA (bDNA) probes.

These SNPs are linked to the following genes and these genes are, accordingly, associated with polymorphism of IVIG resistance in KD: ADTRP (r59380548); KLF6 (r57634); EXOC2 (EXOC2); ZNF438-ZEB1 (s1250301 and r52797915) or MIR548A3-ZPLD1 (r54130857, rs4585205 and r54602399).

Methods for Treating Kd Patients with Ivig Resistance

After identifying KD patients with IVIG resistance based on the methods disclosed herein, adjunctive therapy can be administered with the first does of IVIG to minimize the complication of KD, especially coronary artery lesions.

Non limiting examples of additional therapy include a second dose of IVIG anti-TNF-α agent, corticosteroid, cyclosporin, IL-1 inhibitors (anakinra and canakinumab), cyclophosphamide, Rituximab, Tocilizumab, Pentoxifylline, and plasmapheresis or combination thereof.

The dosage of the adjunctive therapy can be determined by the skilled person in the art according with the age, weight, condition of the subject to be treated, any exiting medical conditions, and on the discretion of medical professionals. The adjunctive therapy can be administered before, simultaneously or after the administration of the first does of IVIG

Kits for Identifying KD Patients with IVIG Resistance

The present invention also provides the use of a kit for identifying a KD patient with IVIG resistance. The kit comprises an agent for detecting the risk allele of at least one SNP selected from rs9380548, rs742490, rs7634, rs1250301, rs2797915, rs4130857, rs4585205, rs4602399, rs4286440, rs16867137 or rs1250302 in the sample of the patient.

Non-limiting examples of the agent include a primer set for isolating and amplifying DNA or a hybridization probe capable of detecting at least one SNP. The term “primer” refers to an oligonucleotide used in a polymerase chain reaction (PCR) reaction. The appropriate primer set may be easily designed by those skilled in the art with reference to the sequences according to an embodiment of the present invention.

In some embodiments, the kit includes a microarray. A microarray is a microscopic, ordered array of nucleic acids, proteins, small molecules, cells or other substances that enables parallel analysis of complex biochemical samples. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

A microarray according to certain embodiments of the present invention includes allele-specific oligonucleotide (ASO) probes (i.e., polynucleotide hybridized with the polynucleotide of SEQ ID NOS:1-11). The ASO probes may be immobilized on a substrate coated with an active group selected among amino-silane, poly-L-lysine and aldehyde. Also, the substrate may be composed of a silicon wafer, glass, quartz, metal or plastic. The method of immobilizing the polynucleotide on the substrate may be either micropipetting using piezoelectric or a method using a pin-shaped spotter

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

### Example 1

### Methods

The DNA of children who have been diagnosed as KD and received IVIG treatment at Chang Gung Memorial Hospital, Kaohsiung Medical Center from the year 2001 to 2007 were collected. The diagnosis of KD was made by a clinician using the criteria for KD proposed by the American Heart Association: fever lasting for >5 days with four of the following criteria: diffuse mucosal inflammation with strawberry tongue and fissure lips, bilateral nonpurulent conjunctivitis, indurative angioedema over the hands and feet, dysmorphic skin rashes, and unilateral cervical lymphadenopathy. Samples from children with acute fever for <5 days were excluded.

Every patient diagnosed as KD was treated with a single dose of IVIG infusion (2 g/kg) within a 12-hour period. Aspirin (3 to 5 mg per kg per day) was given until all signs of inflammation were resolved or regression of CAL under 2-dimensional echocardiography test by visualizing coronary arteries (including left and right) diameter on the parasternal short-axis view of the aorta. Following the Japanese Ministry of Health guidelines, we defined CAL as an increment of the internal diameter of 3 mm (≤5 years old) or 4 mm>5 years old), or 1.5× larger of internal diameter than the adjacent segment. We defined IVIG responsiveness as defervescence within 48 hours after the administration of first IVIG KD patients who had persistence of fever beyond 48 hours were defined as IVIG resistant.

The blood cells of the KD patients were first treated with 0.5% sodium dodecylsulfate lysis buffer and then protease K (1 mg/mL) for 4 hours at 60° C. DNA was extracted using Gentra extraction kit (Qiagen, USA) followed by 70% alcohol precipitation and Gentra Puregene Blood Kit (Qiagen). Single-nucleotide polymorphisms (SNPs) were detected by using Affymetrix Genome-Wide Human SNP Array 6.0 platform (Affymetrix, Inc, USA).

PLINK31 (S. Purcell et al., Am J Hum Genet. 2007; 81:559-575) was applied for quality control of genome-wide scan data. We excluded the SNPs with missing call rates exceeding 1.0%, a P value of Hardy-Weinberg equilibrium <1×1⁰⁻⁰⁵ and minor allele frequency <5.0%. The SNPs reference of Affymetrix Genome-Wide Human SNP Array 6.0 platform was NCBI36 (hg18). CrossMap (Version 0.1.5) was used to lift over data to NCBI37 (hg19). SHAPEIT (O. Delaneau et al., Nat Methods. 2011; 9:179-181.) and IMPUTE2 (BN Howie et al., PLoS Genet. 2009; 5:e1000529) were applied for the haplotype phasing and genotype imputation. HapMap 3 genotype data were incorporate (The International HapMap Consortium. Nature. 2003; 426:789-796) with the Taiwanese data to perform principal component analysis (PCA). PCA was performed by using Genome-wide Complex Trait Analysis, 35 which performed PCA by the same algorithm implemented in EIGENSTRAT and output corresponding eigenvalues and eigenvectors, to identify sample substructure on autosomal genotype data.

To satisfy the 603698 SNPs to filtering criteria, association analysis was performed using the mixed linear model algorithm implemented in Genome-wide Complex Trait Analysis that accounts for the polygenic effect of all SNPs during association test. The fixed effect of all SNPs was calculated by excluding the candidate markers (mixed linear model with candidate marker excluded), which prevented loss of power because of double fitting of the candidate markers. Manhattan plot was plotted by Haploview software (JC Barrett et al., Bioinformatics. 2005; 21:263-265). The residual population stratification was evaluated by calculating genomic inflation λ value and visualized corresponding test statistics using quantile-quantile plot in R (http://www.r-project.org/).

wGRS system (PL. De Jager et al., Lancet Neurol. 2009; 8:1111-1119) was applied to calculate the cumulative effects of candidate SNPs. The allelic odds ratios were natural logarithm transformed to become the weight of each SNP. The wGRS was calculated by multiplying the weight of each SNP ((ln(OR)) by the risk allele number (0, 1, or 2) and taking the sum across at least 8 SNPs, as shown in the following formula:

$$wGRS = {\sum }_{k = 1}^{n} {w}^{k} {X}^{k}$$

wherein n is an integer between 8 to 11, k is SNP, w^{k} is the corresponding weight of SNP (ln(OR)) and Xk is the number of the risk allele (0, 1, or 2). wGRSs of IVIG responders and nonresponders have been compared by Wilcoxon rank-sum test with continuity correction. KD patients were then categorized by corresponding wGRS into 4 groups: group 1 (wGRS<|mean−SD|), group 2 (|mean−SD|≤wGRS<median), group 3 (median≤wGRS<|mean+SD|), and group 4 (wGRS≥|mean+SD|). wGRS was also compared between groups, and relevant statistical parameters were calculated by using group 1 as a reference. The subgroup analysis was further performed to confirm the intragroup difference of wGRS between IVIG responders and nonresponders.

Results

Sample Substructure Evaluation of GWAS:

150 KD patients (n=150) were included in this study. In total, 867 877 SNPs were genotyped in 24 IVIG nonresponders (male=58.33%, age=2.24±2.82 years old) and 126 IVIG responders (male=60.32%, age=2.16±2.41 years old). After marker-level quality control, 264 179 of 867 877 (30.44%) markers were filtered. SNPs were defined to pass quality control with following criteria: minor allele frequency >5.0%, genotyping call rate >99%, and passed Hardy-Weinberg equilibrium test in controls (P<1×10-05). SNPs of 603 698 of 867 877 (69.56%) remained for further analysis. In sample level quality control step, all subjects passed the criteria of sample call rate >99% and were retained for further analysis. PCA was performed to assess sample structure and no significant population stratification was identified. P values from an allelic association test showed no inflation and revealed a low possibility of sample substructure (genomic inflation factor λ value λGC=1.015).

Risk Loci Affecting IVIG Responsiveness:

To identify the risk loci affecting IVIG responsiveness, a mixed linear model association test was performed. Eleven SNPs with P<1×10-05 were identified (Table 2).

TABLE 2
SNPs identified from the KD and non-KD cohorts
that are associated with IVIG resistance.

| | | RA^{a}/ | | | | | % of total |
|---|---|---|---|---|---|---|---|
| Chr. | SNP | NRA^{b} | RAF^{c} | PMLMA^{d} | OR^{e} | Weight^{f} | weight |
| 6 | rs9380548 | A/T | 0.05 | 1.04 × 10⁻⁰⁶ | 1.630504 | 0.488889 | 13.95% |
| 10 | rs7634 | A/T | 0.246667 | 1.56 × 10⁻⁰⁶ | 1.267626 | 0.237146 | 6.77% |
| 6 | rs742490 | G/C | 0.073333 | 2.68 × 10⁻⁰⁶ | 1.489495 | 0.398437 | 11.37% |
| 10 | rs1250301 | G/A | 0.07 | 3.52 × 10⁻⁰⁶ | 1.463538 | 0.380857 | 10.87% |
| 10 | rs2797915 | T/C | 0.07 | 3.52 × 10⁻⁰⁶ | 1.463538 | 0.380857 | 10.87% |
| 3 | rs4130857 | A/G | 0.21 | 5.14 × 10⁻⁰⁶ | 1.265988 | 0.235853 | 6.73% |
| 3 | rs4585205 | T/C | 0.21 | 5.14 × 10⁻⁰⁶ | 1.265988 | 0.235853 | 6.73% |
| 3 | rs4602399 | G/T | 0.21 | 5.14 × 10⁻⁰⁶ | 1.265988 | 0.235853 | 6.73% |
| 3 | rs4286440 | G/T | 0.213333 | 7.09 × 10⁻⁰⁶ | 1.261098 | 0.231983 | 6.62% |
| 2 | rs16867137 | C/T | 0.093333 | 7.57 × 10⁻⁰⁶ | 1.374688 | 0.318227 | 9.08% |
| 10 | rs1250302 | G/A | 0.073333 | 8.11 × 10⁻⁰⁶ | 1.43344 | 0.360077 | 10.28% |

Chr., chromosome location;
^{a}Risk allele,
^{b}Non-risk allele,
^{c}Risk allele frequency in cases and controls.
^{d}PMLMA are calculated by the mixed linear model association test.
^{e}Odds ratio for risk allele.
^{f}Weight is calculated using natural logarithm transformation of odds ratio.

A scoring system based on wGRS proposed by De Jager et al was calculated based on the 11 SNPs in Table 1. The wGRS showed a significant difference (Wilcoxon rank-sum test, P=1.071×10₋₁₀) between IVIG responsive and nonresponsive groups. Patients were categorized into 4 strata: group 1 (wGRS<0.042), group 2 (0.042wGRS<0.636), group 3 (0.636 wGRS<1.681), and group 4 (wGRS1.681). As shown in Table 3, all patients (42 of 42) in group 1 showed good responsiveness to IVIG treatment, whereas 14 of 21 IVIG nonresponders were in the group 4. The results indicated that the patient categorized in group 4 would be estimated to have 164.33 fold higher risk for IVIG resistance compared with those in group 1 (P=8.224×10-10; 95% CI, 8.83-3059.50). The sensitivity and specificity of group 4 versus group 1 were 100% and 85.7%, respectively.

TABLE 2
wGRS Analysis

| | | Nonresponders/ | Fisher's Exact | | | |
|---|---|---|---|---|---|---|
| | wGRS* | Responders | P Value† | OR (95% CI) | Sensitivity | Specificity |
| 4 | ≥1.681 | 14/7 | 8.224 × 10⁻¹⁰ | 164.33 (8.83-3059.50) | 1.000 | 0.857 |
| 3 | ≥0.636-<1.681 | 9/48 | 4.518 × ¹⁰^{^{−03}} | 16.65 (0.94-294.67) | 1.000 | 0.467 |
| 2 | ≥0.042-<0.636 | 1/29 | 1.000 | 4.32 (0.17-109.79) | 1.000 | 0.592 |

| | | | |
|---|---|---|---|
| 1 | <0.042 | 0/42 | (Reference) |

P value and odds ratio (OR) were calculated after Haldane correction. CI indicates confidence interval; and wGRS, weighted genetic risk score.
*Weighted genetic risk score.
†Two-tail P value was calculated by Fisher's exact test.

Further analysis between groups 4 and 1 indicated a positive predictive value of 0.667, negative predictive value of 1.000, positive likelihood ratio of 6.993 (moderate evidence to rule in disease), and negative likelihood ratio of 0 (strong evidence to rule out disease).

ROC curves were constructed to evaluate the performance of wGRS in categorizing KD patients into the IVIG responsive or nonresponsive group. The results indicated that the best predictive value of wGRS was 1.3, and the corresponding specificity and sensitivity were 88.9% and 79.2%, respectively. After adjusted for sex effects, the performance of wGRS remains well, with the best value of 2.8. The corresponding specificity and sensitivity were 81.7% and 79.2%, respectively). Comparison between 2 ROCs showed a P value of 0.10502 (Z statistic of 1.621) under the null hypothesis of a true difference in AUC was equal to 0.

As rs4130857, rs4585205, and rs4602399 on chromosome 3 and rs1250301 and rs2797915 on chromosome 10 are in highly linkage disequilibrium, rs4130857 and rs1250301 were selected (in total 8 SNPs) for the wGRS analysis. The results in Table 3 shows the wGRSs based on 8 SNPs are similar compared to those based on 11 SNPs in Table 2.

TABLE 3
Weighted genetic risk score (wGRS) analysis with using 8 SNPs.

| | | Nonresponders/ | Fisher's Exact | | | |
|---|---|---|---|---|---|---|
| | wGRS^{a} | Responders | P-Value^{b} | OR (95% CI) | Sensitivity | Specificity |
| 4 | ≥1.027 | 16/6 | 8.224 × 10⁻¹⁰ | 220.85 (11.77-4143.31) | 1.000 | 0.878 |
| 3 | ≥0.237 to <1.027 | 7/62 | 0.053 | 10.44 (0.58-187.63) | 1.000 | 0.410 |
| 2 | ≥0.050 to <0.237 | 1/15 | 0.074 | 8.42 (0.33-217.74) | 1.000 | 0.741 |

| | | | |
|---|---|---|---|
| 1 | <0.050 | 0/43 | (Reference) |

^{a}Weighted genetic risk score.
^{b}Two-tail P-value was calculated by Fisher's exact test. P-value and odds ratio were calculated after Haldane's correction.

What is claimed is:

1. A method for treating Kawasaki Disease in a subject, comprising
(a) detecting the presence of the risk allele of at least one single nucleotide polymorphism (SNP) selected from rs9380548, rs742490, rs7634, rs1250301, rs2797915, rs4130857, rs4585205, rs4602399, rs4286440, rs16867137 or rs1250302 in the sample of the subject;
(b) identifying the subject with at least one risk allele in step (a) as resistant to IVIG; and
(c) administering a first dose of IVIG and an adjunctive therapy to the subject.

2. The method of claim 1, wherein the adjunctive therapy is a second dose of IVIG, anti-TNF-α agent, corticosteriod, cyclosporin, IL-1 inhibitors (anakinra and canakinumab), cyclophosphamide, Rituximab, Tocilizumab, Pentoxifylline, and plasmapheresis or combination thereof.

3. The method of claim 2, wherein the adjunctive therapy is the second dose of IVIG.

4. A method for treating Kawasaki Disease in a subject, comprising the steps of
(a) detecting the presence of the risk allele of the single nucleotide polymorphisms (SNPs) in the sample of the subject, said SNPs are selected from:
(i) rs9380548;
(ii) rs742490;
(iii) rs7634;
(iv) at least one SNP selected from rs1250301 or rs2797915;
(v) at least one SNP selected from rs4130857, rs4585205 or rs4602399;
(vi) rs4286440;
(vii) rs16867137; and
(viii) rs1250302;
(b) calculating a weighted genetic risk score (wGRS) based on the following formula:
$$wGRS = {\sum }_{k = 1}^{n} {w}^{k} {X}^{k}$$
wherein n is an integer between 8 to 11, k is the SNP of step (a), w^{k} is the corresponding weight of SNP (ln(OR)) and X^{k} is the number of the risk allele of the SNP (0, 1, or 2);
(c) identifying the subject as resistant to IVIG if the wGRS in step (b) is equal to or higher than 0.01; and
(d) administering a first dose of IVIG and an adjunctive therapy to the subject.

5. The method of claim 4, wherein the adjunctive therapy is a second dose of IVIG, anti-TNF-α agent, corticosteroid, cyclosporin or combination thereof.

6. The method of claim 4, wherein the wGRS is equal to or higher than 0.04.

7. A method to identify a Kawasaki Disease subject resistant to IVIG, comprising the steps of:
(a) detecting the presence of the risk allele of the single nucleotide polymorphisms (SNPs) in the sample of the subject, said SNPs are selected from:
(i) rs9380548;
(ii) rs742490;
(iii) rs7634;
(iv) at least one SNP selected from rs1250301 or rs2797915;
(v) at least one SNP selected from rs4130857, rs4585205 or rs4602399;
(vi) rs4286440;
(vii) rs16867137; and
(viii) rs1250302;
(b) calculating a weighted genetic risk score (wGRS) based on the following formula:
$$wGRS = {\sum }_{k = 1}^{n} {w}^{k} {X}^{k}$$
wherein n is an integer between 8 to 11, k is the SNP of step (a), w^{k} is the corresponding weight of SNP (ln(OR)) and X^{k} is the number of the risk allele of the SNP (0, 1, or 2)
(c) identifying the subject as IVIG resistant if the wGRS in step (b) is equal to or higher than 0.01.

8. The method of claim 7, wherein the wGRS is equal to or higher than 0.04.
